(19) United States
(12) Patent Application Publication
(10) Pub. No.: US 2014/0051662 A1
(43) Pub. Date: Feb. 20, 2014

(54) TREATMENT OF MULTIPLE MYELOMA WITH MASITINIB

(71) Applicants: Alain Moussy, Paris (FR); Jean-Pierre Kinet, Aix-En-Provence (FR)

(72) Inventors: Alain Moussy, Paris (FR); Jean-Pierre Kinet, Aix-En-Provence (FR)

(73) Assignee: AB SCIENCE, Paris (FR)

(21) Appl. No.: 14/008,962

(22) PCT Filed: Apr. 4, 2012
(86) PCT No.: PCT/EP12/56226

§ 371 Date: Oct. 31, 2013

Related U.S. Application Data
(60) Provisional application No. 61/473,380, filed on Apr. 8, 2011.

(51) Int. Cl. A61K 31/496; A61K 45/06; A61K 31/573; A61K 31/10; A61K 31/69

(52) U.S. Cl. CPC A61K 31/496; A61K 31/10; A61K 31/69; A61K 31/573; A61K 45/06
USPC 514/64; 514/253.1; 514/171

(57) ABSTRACT

The present invention relates to the treatment of multiple myeloma, especially for the treatment of those patients with refractory or first relapsed multiple myeloma, and in particular patients with t(4;14) multiple myeloma, comprising administration of a tyrosine kinase inhibitor or a mast cell inhibitor, especially masitinib or a pharmaceutically acceptable salt thereof, administered in association with an additional care in multiple myeloma; for example, autologous stem-cell transplantation, targeted therapies, anti-myeloma agents such as alkylating agents, corticosteroids, or immunomodulatory agents, including bortezomib, lenalidomide, and dexamethasone.

The present invention relates to the treatment of multiple myeloma, especially for the treatment of refractory or first relapsed multiple myeloma, and in particular patients with t(4;14) multiple myeloma, comprising administration of a tyrosine kinase inhibitor or a mast cell inhibitor, especially masitinib or a pharmaceutically acceptable salt thereof, administered in association with an additional care in multiple myeloma; for example, autologous stem-cell transplantation, targeted therapies, anti-myeloma agents, including: alkylating agents, corticosteroids, or immunomodulatory agents, including bortezomib, lenalidomide, and dexamethasone.

### BACKGROUND OF THE INVENTION

### Multiple Myeloma Overview

Multiple myeloma (also known as myeloma or plasma cell myeloma) is a progressive hematologic disease, characterized by excessive numbers of abnormal plasma cells in the bone marrow and overproduction of intact monoclonal immunoglobulin. It is thought that multiple myeloma usually arises from a common benign plasma cell tumor called Monoclonal Gammopathy of Undetermined Significance (MGUS). Multiple myeloma accounts for 1% of all cancers and 10% of all hematologic malignancies (making it the second most common hematological malignancy) and 2% of all cancer deaths. In multiple myeloma patients, mutated plasma cells grow unregulated by the processes that normally control cell division and death. The interaction of cytokines such as interleukin (IL)-6 and tumor necrosis factor (TNF), stimulate the growth of myeloma cells and inhibit apoptosis, leading to proliferation of myeloma cells. Myeloma plasma cells have specific adhesion molecules on their surface allowing them to attach to bone marrow stromal cells. Thus, myeloma cells traveling through the bloodstream can collect in the bone marrow where they interfere with cells in the bone that produce white and red blood cells and platelets, often causing anemia and a decreased immune function. The overgrowth of plasma cells in the bone marrow often leads to structural bone damage, resulting in bone pain and fractures. Myeloma cells also produce abnormal antibodies that cannot effectively fight infection. As tumors grow they invade the hard, outer part of the bone, eventually spreading into the bone marrow of all the large bones of the body, with myeloma cells found in multiple sites throughout the bone marrow. The diagnosis of multiple myeloma requires (i) 10% or more clonal plasma cells on bone marrow examination or a biopsy-proven plasmacytoma, plus (ii) evidence of end-organ damage felt to be related to the underlying plasma cell disorder.

Multiple myeloma is characterized by marked genetic heterogeneity with two broad genetic subtypes defined by chromosome number, namely, hyperdiploid multiple myeloma and nonhyperdiploid multiple myeloma. The latter is associated with primary IgH translocations including (11;14)(q13;q32) and t(4;14)(p16;q32), respectively representing 20% and 15% of multiple myeloma cases. It has been shown that multiple myeloma patients with the chromosomal abnormality t(4;14) have a poor prognosis and poor overall survival with aggressive relapse and short remission times even following a positive response to stem cell transplantation.

### Treatment of Multiple Myeloma

Although there is no cure for multiple myeloma the overall survival in myeloma has improved significantly in the last decade with the emergence of drugs such as thalidomide (Thalomid®), bortezomib (Velcade®), and lenalidomide (Revlimid®). The major regimens used for therapy are listed in Table 1. It is possible that development of such novel regimens might obviate the need for autologous stem-cell transplantation (ASCT) in a sizeable proportion of patients. Moreover, the emergence of new therapeutic options and regimens appears likely to significantly alter the first-line treatment paradigm for patients with multiple myeloma. Such new treatments target not only to the malignant plasma cell but also act on crucial pathways necessary for multiple myeloma cell survival, proliferation, migration, and drug resistance. For example, interactions of multiple myeloma cells with their microenvironment including bone marrow stromal cells, extracellular matrix proteins, osteoclasts, endothelial cells, immunocompetent cells, cytokines, and growth factors. These novel agents may be either small molecules or monoclonal antibodies, collectively referred to as ‘targeted therapies’.

TABLE 1
Major treatment regimes in Multiple Myeloma (taken from Rajkumar SV. Multiple
myeloma: 2011 update on diagnosis, risk-stratification, and management. Am. J. Hematol.
86: 57-65, 2011).

| Regimen | Usual dosing schedule^{a} |
|---|---|
| Melphalan-Prednisone (7-day schedule) [1] | Melphalan 8-10 mg oral days 1-7 |
| | Prednisone 60 mg/day oral days 1-7 |
| | Repeated every 6 weeks |
| Thalidomide-Dexamethasone [55, 56]^{b} | Thalidamide 200 mg oral days 1-28 |
| | Dexamethasone 40 mg oral days 1, 8, 15, and 22 |
| | Repeated every 4 weeks |
| Lenalidomide-Dexamethasone [57] | Lenalidomide 25 mg oral days 1-21 every 28 days |
| | Dexamethasone 40 mg oral days 1, 8, 15, 22 every 28 days |
| | Repeated every 4 weeks |
| Bortezomib-Dex [58]^{b} | Bortezomib 1.3 mg/m² intravenous days 1, 8, 15, and 22 |
| | Dexamethasone 20 mg on day of and day after bortezomib (or 40 mg days 1, 8, 15, and 22) |
| | Repeated every 4 weeks |
| Melphalan-Prednisone-Thalidomide [59, 60] | Melphalan 0.25 mg/kg oral days 1-4 (use 0.20 mg/kg/day oral days 1-4 in patients over the |
| | age of 75) |
| | Prednisone 2 mg/kg oral days 1-4 |
| | Thalidomide 100-200 mg oral days 1-28 (use 100 mg dose in patients > 75) |
| | Repeated every 6 weeks |
| Bortezomib-Melphalan-Prednisone [61-63]^{b} | Bortezomib 1.3 mg/m² intravenous days 1, 8, 15, and 22 |
| | Melphalan 9 mg/m² oral days 1-4 |
| | Prednisone 60 mg/m² oral days 1-4 |
| | Repeated every 35 days |
| Bortezomib-Thalidomide-Dexamethasone | Bortezomib 1.3 mg/m² intravenous days 1, 8, 15, and 22 |
| [64, 65]^{b} | Thalidomide 100-200 mg oral days 1-21 |
| | Dexamethasone 20 mg on day of and day after bortezomib (or 40 mg days 1, 8, 15, and 22) |
| | Repeated every 4 weeks × 4 cycles as pretransplant induction therapy |
| Cyclophosphamide-Bortezomib-Dexamethasone | Cyclophosphamide 300 mg/m² orally on days 1, 8, 15, and 22 |
| (CyBorD) [66, 67]^{b} | Bortezomib 1.3 mg/m² intravenously days 1, 8, 15, and 22 |
| | Dexamethasone 40 mg orally on day on days 1, 8, 15, and 22) |
| | Repeated every 4 weeks^{c} |
| Bortezomib-Lenalidomide-Dexamethasone | Bortezomib 1.3 mg/m² intravenous days 1, 8, and 15 |
| [67, 68]^{b} | Lenalidomide 25 mg oral days 1-14 |
| | Dexamethasone 20 mg on day of and day after bortezomib (or 40 mg days 1, 8, 15, and 22) |
| | Repeated every 3 weeks^{d} |

^{a}All doses need to be adjusted for performance status, renal function, blood counts, and other toxicities.
^{b}Doses of dexamethasone and/or bortezomib reduced based on subsequent data showing lower toxicity and similar efficacy with reduced doses.
^{c}Omit day 22 dose if counts are low or when the regimen is used as maintenance therapy; when used as maintenance therapy for high-risk patients, delays can be instituted between cycles.
^{d}Omit day 15 dose if counts are low or when the regimen is used as maintenance therapy; when used as maintenance therapy for high-risk patients, lenalidomide dose may be decreased to 10-15 mg per day, and delays can be instituted between cycles as done in total therapy protocols [67, 70].

Current standards of care for first-line treatment of multiple myeloma are evolving rapidly due to emerging regimens based upon new agents and their unique mechanisms of action. For example, bortezomib is a targeted therapy, specifically a proteasome inhibitor, which causes cancer cells to die by blocking the action of proteasome. It is approved for people with newly diagnosed and previously treated myeloma (i.e. first-line and second-line treatment). The mechanism of action of thalidomide and lenalidomide is unclear, but both drugs possess immunomodulatory properties. Thalidomide is administered orally and is FDA approved for the treatment of newly diagnosed multiple myeloma. Lenalidomide is chemically similar to thalidomide and is also administered orally. It is approved for people with previously treated myeloma, but is also often used in people with newly diagnosed disease. Additionally, there are several standard chemotherapeutic agents used for treatment of multiple myeloma, including the commonly used alkylating agents melphalan (Alkeran®) and cyclophosphamide (Cytoxan®), as well as vincristine (Oncovin®), doxorubicin (Adriamycin®), and liposomal doxorubicin (Doxil®). Standard treatment also includes the use of corticosteroids such as prednisone and dexamethasone (Decadron®). Dexamethasone can be given alone or in combination with other chemotherapeutic drugs, including most commonly with thalidomide, lenalidomide, bortezomib, or a combination of doxorubicin and vincristine. Other nascent treatments include the immodulator pomalidomide, the keto-epoxide tetrapeptide proteasome carfilzomib, the anti-CS-1 antibody elotuzumab, and histone deacetylase inhibitors of vorinostat and panabinostat.

The approach to treatment of symptomatic newly diagnosed multiple myeloma (first-line treatment) is dictated by eligibility for ASCT and risk-stratification. Although staging using the Durie-Salmon staging or the International Staging System provide prognostic information for counseling, it is not useful for making therapeutic choices. The International Myeloma Working Group has proposed a risk-stratification model that relies on a number of independent molecular cytogenetic markers and is useful for both counseling and therapeutic decision making [Fonseca R et al. International Myeloma Working Group molecular classification of multiple myeloma: Spotlight review. Leukemia 2009; 23:2210-2221]. Using such a model, newly diagnosed myeloma is stratified into standard-risk and high risk disease. Patients with 17p deletion, t(4;14), t(14;16), t(14;20), and karyotypic deletion 13 or hypodiploidy are considered to have high-risk myeloma. All others are considered to have standard-risk disease. In general, standard-risk patients are treated with nonalkylator-based therapy such as lenalidomide plus low-dose dexamethasone followed by autologous stem-cell transplantation (ASCT). High-risk patients are treated with a bortezomib-based induction followed by ASCT and then bortezomib-based maintenance [Rajkumar S V. Multiple myeloma: 2011 update on diagnosis, risk-stratification, and management. Am. J. Hematol. 86:57-65, 2011].

Angiogenesis, or neovascularization, is the formation of new blood vessels from pre-existing vessels, and is a mechanism that plays an important role in the pathogenesis and progression of multiple myeloma. Although the strategy of anti-angiogenic treatment of multiple myeloma has been demonstrated in the preclinical setting with various targeted agents, predominantly inhibitors of VEGFR, only moderate success has been achieved at clinical trial [Ribatti D. Pharmaceuticals 2010, 3, 1225-1231]. The main problem in the development of anti-angiogenic agents is that multiple angiogenic molecules may be produced by tumors, and tumors at different stages of development may depend on different angiogenic factors for their blood supply. Therefore, blocking a single angiogenic molecule will have little or no impact on tumor growth and over time will be prone to the development of acquired resistance.

Relapsed myeloma, also called recurrent myeloma, is multiple myeloma that returns after a successful course of treatment. Most people who are treated for multiple myeloma eventually experience a relapse of the disease. The International Myeloma Working Group (IMWG) Uniform Response Criteria for Multiple Myeloma [Durie et al. Leukemia. 20, 1467-1473, 2006] define clinical relapse to require one or more of:

Direct indicators of increasing disease and/or end organ dysfunction (CRAB features).
1. Development of new soft tissue plasmacytomas or bone lesions.
2. Definite increase in the size of existing plasmacytomas or bone lesions. A definite increase is defined as a 50% (and at least 1 cm) increase as measured serially by the sum of the products of the cross-diameters of the measurable lesion.
3. Hypercalcemia (>11.5 mg/dL) [2.65 mmol/L].
4. Decrease in hemoglobin of >2 g/dL [1.25 mmol/L].
5. Rise in serum creatinine by 2 mg/dL or more [177 mmol/L or more].

In some cases none of the currently available therapies adequately slow the cancer cells from multiplying. This is known as refractory myeloma, multiple myeloma that does not respond to treatment—whether initial therapy or therapy for recurrent disease. Relapsed or refractory multiple myeloma is usually identified on routine surveillance performed during treatment or after the completion of therapy. In general, the management of refractory disease or indolent (non-aggressive) relapse can be treated first with lenalidomide, bortezomib, or alkylators plus low-dose corticosteroids. Patients with more aggressive relapse often require therapy with a combination of multiple active agents [Rajkumar S V Am. J. Hematol. 86:57-65, 2011].

Adjuvant therapy or maintenance therapy may be given to multiple myeloma patients following surgery or following a significant response to first-line treatment (i.e. cancer remission) in an attempt to prevent relapse. For example, thalidomide has shown modest benefit as maintenance therapy, lenalidomide has shown improvement in post-ASCT maintenance therapy and may be important for those patients who fail to achieve very good partial response, and bortezomib-based maintenance approaches may be important in high-risk patients.

Beyond the already developed therapeutic strategies, there exists an imperative need to develop less toxic and more efficient treatment strategies that improve the clinical management and prognosis of patients afflicted with this disease. Several reasons can be given for this:
The continuing poor prognosis and lack of effective treatments for multiple myeloma, in particular for refractory or relapsed patients, highlights an unmet medical need.
None of the available drugs cure or completely stop the disease process and in certain refractory populations are not particularly effective in controlling the disease.
Almost all patients with multiple myeloma eventually relapse and in general the remission duration in relapsed myeloma decreases with each regimen.
Given that some patients respond positively to first-line treatments but then inevitably relapse, there is a need to develop adjuvant therapies and maintenance strategies that slow or prevent relapse after remission.
Clinical trials involving anti-angiogenic agents, such as anti-VEGF agents, induce only modest improvement, with the up-regulation of other pro-angiogenic cytokines reducing their effectiveness.
Important adverse events are reported for existing treatments.

### AIMS OF THE INVENTION

The invention aims to solve the technical problem of providing an active ingredient for the treatment of multiple myeloma in a human patient, and in particular an effective treatment for refractory or first-relapse multiple myeloma.

The invention also aims to solve the technical problem of providing an active ingredient for the effective treatment of t(4:14) multiple myeloma, that is to say, those patients who bear the t(4:14) translocation.

The invention also relates to the treatment of such a disease in a human patient, regardless of said patient's status of fibroblast growth factor receptor 3 (FGFR3) expression, that is to say, for patients who are classified as either FGFR3 positive or FGFR3 negative.

The invention aims to provide an efficient treatment for such a disease at an appropriate dose, route of administration and daily intake.

The invention also aims to solve the technical problem of providing an active ingredient that improves prior art methods for the treatment of multiple myeloma.

The invention also aims to solve the technical problem of providing an active ingredient that promotes consolidation of therapeutic-induced remission and acts as an adjuvant or maintenance therapy in an attempt to prevent a relapse.

### SUMMARY OF THE INVENTION

The invention relates to tyrosine kinase inhibitor or a mast cell inhibitor, especially masitinib or a pharmaceutically acceptable salt thereof, for the treatment of multiple myeloma, especially for the treatment of refractory or first relapsed multiple myeloma, wherein said inhibitor is to be administered to patients in need thereof, and in particular patients with t(4;14) multiple myeloma, in association with an additional care in multiple myeloma; for example, autologous stem-cell transplantation, targeted therapies, anti-myeloma agents, including: alkylating agents, corticosteroids, or immunomodulatory agents.

The invention also relates to a method of treatment of multiple myeloma, especially for the treatment of refractory or first relapsed multiple myeloma, wherein a tyrosine kinase inhibitor or a mast cell inhibitor, especially masitinib or a pharmaceutically acceptable salt thereof, is to be administered to patients in need thereof, and in particular patients with t(4;14) multiple myeloma, in association with an additional care in multiple myeloma; for example, autologous stem-cell transplantation, targeted therapies, anti-myeloma agents, including: alkylating agents, corticosteroids, or immunomodulatory agents.

In one embodiment, the invention relates to a method of treatment of multiple myeloma, especially for the treatment of refractory or first relapsed multiple myeloma, and in particular patients with t(4;14) multiple myeloma, wherein a tyrosine kinase inhibitor, especially masitinib or a pharmaceutically acceptable salt thereof, is a dual c-Kit/FGFR3 inhibitor.

In another embodiment, the invention relates to a method of treatment of multiple myeloma, especially for the treatment of refractory or first relapsed multiple myeloma, and in particular patients with t(4;14) multiple myeloma, wherein a tyrosine kinase inhibitor or a mast cell inhibitor, especially masitinib or a pharmaceutically acceptable salt thereof, is an inhibitor of c-Kit, Lyn, Fyn, and platelet-derived growth factor receptor (PDGFR) kinase activity.

In another embodiment, the invention also relates to a method of treatment of multiple myeloma, especially for the treatment of refractory or first relapsed multiple myeloma, wherein a tyrosine kinase inhibitor or a mast cell inhibitor, especially masitinib or a pharmaceutically acceptable salt thereof, is to be administered to patients in need thereof, and in particular patients with t(4;14) multiple myeloma, in combination with at least one targeted therapy, alkylating agent, corticosteroid, or immunomodulatory agent, including, but not limited to: bortezomib, dexamethasone, thalidomide, lenalidomide, doxorubicin, vincristine, melphalan, cyclophosphamide, pomalidomide, carfilzomib, elotuzumab, vorinostat, and panabinostat.

In yet another embodiment, the invention also relates to a method of treatment of multiple myeloma, especially for the treatment of refractory or first relapsed multiple myeloma, and in particular patients with t(4;14) multiple myeloma, wherein a tyrosine kinase inhibitor or a mast cell inhibitor, especially masitinib or a pharmaceutically acceptable salt thereof, acts as an adjuvant or maintenance therapy to prevent relapse following treatment-induced remission or post-autologous stem-cell transplantation.

### DESCRIPTION OF THE INVENTION

Role of FGFR3, c-Kit, and PDGFR in Multiple Myeloma

The transformation of normal cells into tumoral cells often results from aberrant regulations of their proliferation by growth factor receptors. In the case of multiple myeloma, both FGFR3 and c-Kit are aberrantly expressed in a subset of tumoral cells while normal plasma cells do not express either of these receptors.

Aberrant expression of FGFR3 (a tyrosine kinase receptor which belongs to the high-affinity FGFR family) is found in the majority of patients with the t(4,14) translocation. FGF-binding induces cell proliferation and differentiation through dimerization of FGFR3, tyrosine auto-phosphorylation and activation of the MAP kinase, PLC gamma and PI3 kinase pathways. Ectopic expression of FGFR3 in plasma cells seems to have a transforming activity including IL-6-independent growth, resistance to dexamethasone-induced apoptosis [Pollett J B et al., Blood. 2002; 100(10):3819-21], and resistance to bortezomib-induced apoptosis [Guan M et al., Anticancer Res. 2009; 29(1):1-9]. Thus, drugs capable of inhibiting FGFR3 may be of therapeutic benefit in the treatment of this subpopulation of multiple myeloma. This principle has been demonstrated in-vitro by a number of selective FGFR3 inhibitors with reports of induced cytotoxic responses in FGFR3-expressing myeloma cells. However, these pre-clinical findings have not translated into clinical success, indicating that targeting FGFR3 alone is not sufficiently efficient in vivo. Therefore, FGFR3 represents a potential therapeutic target for t(4,14) multiple myeloma, but only in concert with dual or multiple kinase targeting or in combination with other anti-myeloma agents.

A subset of patients (30%) with multiple myeloma expresses c-Kit (CD117) in tumor plasma cells. Two different isoforms of c-Kit, characterized by the presence or absence of the tetrapeptide sequence GNNK in the extracellular domain, have been described (termed c-Kit GNNK-positive or c-Kit GNNK-negative). Montero et al. investigated the impact of c-Kit on the action of drugs commonly used in the treatment of multiple myeloma (Montero et al., 2008, hematological 93(6):851-8). Results indicated that c-Kit expression in multiple myeloma cells is functional, and is coupled to survival pathways that may modulate cell death in response to therapeutic compounds used in the treatment of this disease (both GNNK positive and GNNK negative), with cells expressing the GNNK-negative form shown to be more resistant to the anti-myeloma action of bortezomib and melphalan. Moreover, the addition of Stem Cell Factor (SCF) partially protected against dexamethasone-induced apoptosis of cells expressing either of the two c-Kit isoforms. Thus, drugs capable of inhibiting c-Kit may be of therapeutic benefit in the treatment of multiple myeloma by acting in a synergistic manner with anti-myeloma agents.

PDGFR is also associated with the pathogenesis of multiple myeloma. Coluccia et al. identified PDGFR-beta as being constitutively activated in plasma cells and endothelial cells isolated from multiple myeloma patients. In vitro studies showed that the PDGF-BB/PDGFR kinase axis promoted multiple myeloma tumor growth and vessel sprouting by activating ERK1/2, AKT, and the transcription of multiple myeloma endothelial cell-released pro-angiogenic factors, such as vascular endothelial growth factor (VEGF) and interleukin-8 (IL-8) [Coluccia A M L et al., Blood. 2008; 112:1346-1356]. Thus, targeting of PDGF signaling pathways could promote an anti-tumor/vessel effect in multiple myeloma. In addition, higher serum PDGF-BB receptor levels have been associated with melphalan-resistant multiple myeloma patients and it has been shown that PDGF-BB modulates the expression of c-myc protein [Greco C et al., Int J Immunopathol Pharmacol. 2006; 19(1):67-79].

### Role of Mast Cells in Multiple Myeloma

Mast cells are predominantly found in tissues at the interface between the host and the external environment, such as lung, connective tissue, lymphoid tissue, gut mucosa, and skin. Immature mast cells progenitors circulate in the bloodstream and differentiate in tissues. These differentiation and proliferation processes are influenced by cytokines, notably SCF. The SCF receptor is encoded by the proto-oncogene c-Kit. It has been shown that SCF regulates the migration, maturation, proliferation, and activation of mast cells in vivo—injection of recombinant SCF into rodents, primates, or humans, results in an increase in mast cell numbers at both the site of injection and at distant sites.

Mast cells are characterized by their heterogeneity, not only regarding tissue location and structure but also at functional and histochemical levels. Mast cell activation is followed by the controlled release of a variety of mediators that are essential for the defense of the organism against invading pathogens. By contrast, in the case of hyperactivation of mast cells, uncontrolled hypersecretion of these mediators is deleterious for the body. Mast cells produce a large variety of mediators categorized here into three groups:
Preformed granule-associated mediators (histamines, proteoglycans, and neutral proteases);
Lipid-derived mediators (prostaglandins, thromboxanes and leucotrienes);
Various cytokines (including the interleukins: IL-1, IL-2, IL-3, IL-4, IL-5, IL-6, IL-8, IL-17, and tumor necrosis factor alpha TNF-α, GM-CSF, MIP-1α, MIP-1β and IFN-γ).

Human mast cells constitutively express a number of receptors for different biological molecules. Among these receptors, whose ligation induces the activation of mast cells, the best known is the high affinity receptor for IgE (FcεRI). Binding of IgE-multivalent antigen complexes to FcεRI leads to receptor aggregation and internalization, signaling, and degranulation. This can be accompanied by the transcription of cytokine genes, thus, perpetuating the inflammatory response. Moreover, triggering of mast cells leads to the secretion of diverse pre-formed and/or de novo synthesized mediators, such as vasoactive amines (histamine, serotonin), sulfated proteoglycans, lipid mediators (prostaglandin D2, leucotrienes), growth factors, proteases, cytokines and chemokines as described previously. These mediators can, alone or in synergy with macrophage-derived and T cell-derived cytokines, generate a complex inflammatory response and induce the recruitment and activation of inflammatory cells to the site of degranulation.

Multiple myeloma in humans is frequently associated with mast cell infiltration and neovascularization, which correlate directly with disease severity. Vascular density and degree of mast cell infiltration in myeloma tissues have been found to directly correlate with each other and with disease activity and grade. Research by Vacca et al. concluded that mast cells contribute to the vascular phase via angiogenic factors in their secretory granules, suggesting a role for mast cell-induced angiogenesis in promoting growth of multiple myeloma [Vacca A. et al. 2001. Semin. Oncol. 28:543-550]. In a study by Ribatti, et al. it was shown that bone marrow angiogenesis and mast cell counts were highly correlated in patients with non-active and active multiple myeloma and in those with MGUS, and that both parameters increased simultaneously in active multiple myeloma [Ribatti, et al 1999. Br. J. Cancer. 79:451-455]. This suggests that active multiple myeloma represent the vascular phase of plasma cell tumors, whereas nonactive multiple myeloma and MGUS represent the prevascular phase. Also, the switch in MGUS and nonactive multiple myeloma may be induced by tumor plasma cells via secretion of angiogenic factors. It is known that mast cells produce a variety of multifunctional cytokines and growth factors, such as IL-1, IL-6 and IL-8, TNF-α, granulocyte macrophage colony-stimulating factor (GM-CSF), transforming growth factor beta (TGF-β), basic fibroblast growth factor (FGF-2), and VEGF-A, which may contribute to angiogenesis and chemoresistance in active multiple myeloma. Ribatti concluded that an increasing number of mast cells are recruited and activated by more malignant plasma cells in active multiple myeloma, and that angiogenesis in this disease phase may be mediated, at least in part, by angiogenic factors contained in their secretory granules. In addition, a study performed by Nakayama et al. [J Clin Invest. 2004; 114:1317-1325] demonstrated a clear role for mast cell derived angiopoeitin 1 (Ang-1) in a mouse model of multiple myeloma. Ang-1 and VEGF-A expression were found in murine mast cells, along with high levels of VEGF-A expression by two plasmacytoma cell lines. In implanted Matrigel plugs, vascularization was induced in the presence of bone marrow-derived mast cells and plasmacytoma cells, with little angiogenesis in the presence of each cell type individually and this effect was inhibited by addition of antibodies against Ang-1, VEGF-A or both. Following injection into Balb/c nude mice, mast cells greatly increased both plasmacytoma tumor size and vascularization. These results demonstrated that mast cell-derived Ang-1 plays a potent role in promoting angiogenesis in multiple myeloma, and provides evidence supporting a causal relationship between inflammation and tumor growth.

Another indication of mast cell involvement in the events mediating transformation in MGUS and nonactive multiple myeloma patients to active multiple myeloma is revealed by research showing that Myc is activated in the majority of newly diagnosed multiple myeloma patients but not in MGUS [Chng W J et al. Blood 2009 114: Abstract 834]. The Myc protein is a transcription factor that regulates a variety of cellular processes including cell growth and proliferation, cell-cycle progression, transcription, differentiation, apoptosis, and cell motility. Although the connection between mast cells and Myc activation in multiple myeloma has received little attention, a comparable relationship has been observed in pancreatic cancer. Soucek et al. reported that Myc activation in mice rapidly induced the release of mast-cell chemoattractants with a consequence of rapid mast cells recruitment to the tissue surrounding the pancreas [Soucek L et al. Nat Med 2007, 13, 1211-8]. Mast cells were the only inflammatory cells increased in the vicinity of tumor cells at this early time (within 24 hours), and their infiltration correlated with the expansion of islet tumors. The presence of mast cells was also required for the maintenance of established tumors in this animal model. Treatment of the mice with the mast-cell stabilizer disodium cromoglycate (cromolyn) led to tumor hypoxia and tumor-cell apoptosis. Moreover, tumors could not be induced in mast-cell-deficient mice. Myc activation was not affected in the mast cell deficient mice, indicating that the absence of mast cells, rather than aberrant Myc function, prevented the growth of pancreatic tumors and that this in turn was due to a defect in tumor angiogenesis. Therefore, it is plausible that Myc activation associated with the onset of active multiple myeloma may be a significant driving factor in mast cell recruitment to the tumor site and subsequent angiogenesis necessary for disease progression.

Thus, inhibition of mast cell survival and activation might act as a new target for adjuvant treatment of multiple myeloma, in part through the diminished release of multiple angiogenic factors contained in their secretory granules. This might restrain disease progression in its own right or help produce a microenvironment conducive for other anti-myeloma agents to work more efficiently. The transforming event of Myc activation to active multiple myeloma and possible initiation of rapid mast cell recruitment, suggests any anti-mast cell strategy would optimally be applied at an early stage of the disease.

### Tyrosine Kinase Inhibitors (Compounds of the Invention)

Tyrosine kinases are receptor type or non-receptor type proteins, which transfer the terminal phosphate of ATP to tyrosine residues of proteins thereby activating or inactivating signal transduction pathways. These proteins are known to be involved in many cellular mechanisms, which in case of disruption, lead to disorders such as abnormal cell proliferation and migration as well as inflammation. A tyrosine kinase inhibitor is a drug that inhibits tyrosine kinases, thereby interfering with signaling processes within cells. Blocking such processes can stop the cell growing and dividing.

Such Tyrosine Kinase Inhibitors are optionally substituted 2-(3-aminoaryl)amino-4-aryl-thiazoles preferably of the following formula I.

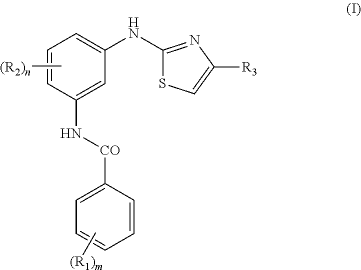

Wherein:

R1 and R2 are selected independently from hydrogen, halogen, a linear or branched alkyl, cycloalkyl group containing from 1 to 10 carbon atoms, trifluoromethyl, alkoxy, cyano, amino, alkylamino, dialkylamino, solubilizing group.
m is 0-5 and n is 0-4.
R3 is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, cyano and alkoxy;
(ii) a heteroaryl group such as 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.

Unless otherwise specified, the below terms used herein are defined as follows:

As used herein, the term an “aryl group” means a monocyclic or polycyclic-aromatic radical comprising carbon and hydrogen atoms. Examples of suitable aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or more substituents.

In one embodiment, the aryl group is a monocyclic ring, wherein the ring comprises 6 carbon atoms, referred to herein as “(C6)aryl.”

As used herein, the term “alkyl group” means a saturated straight chain or branched non-cyclic hydrocarbon having from 1 to 10 carbon atoms. Representative saturated straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. Alkyl groups included in compounds of this invention may be optionally substituted with one or more substituents.

As used herein, the term “alkoxy” refers to an alkyl group which is attached to another moiety by an oxygen atom. Examples of alkoxy groups include methoxy, isopropoxy, ethoxy, tert-butoxy, and the like. Alkoxy groups may be optionally substituted with one or more substituents.

As used herein, the term “heteroaryl” or like terms means a monocyclic or polycyclic heteroaromatic ring comprising carbon atom ring members and one or more heteroatom ring members (such as, for example, oxygen, sulfur or nitrogen). Typically, a heteroaryl group has from 1 to about 5 heteroatom ring members and from 1 to about 14 carbon atom ring members. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzo(b)thienyl. A heteroatom may be substituted with a protecting group known to those of ordinary skill in the art, for example, the hydrogen on a nitrogen may be substituted with a tert-butoxycarbonyl group. Heteroaryl groups may be optionally substituted with one or more substituents. In addition, nitrogen or sulfur heteroatom ring members may be oxidized. In one embodiment, the heteroaromatic ring is selected from 5-8 membered monocyclic heteroaryl rings. The point of attachment of a heteroaromatic or heteroaryl ring to another group may be at either a carbon atom or a heteroatom of the heteroaromatic or heteroaryl rings.

The term “heterocycle” as used herein, refers collectively to heterocycloalkyl groups and heteroaryl groups.

As used herein, the term “heterocycloalkyl” means a monocyclic or polycyclic group having at least one heteroatom selected from O, N or S, and which has 2-11 carbon atoms, which may be saturated or unsaturated, but is not aromatic. Examples of heterocycloalkyl groups including (but not limited to): piperidinyl, piperazinyl, 2-oxopiperazinyl, 2-oxopiperidinyl, 2-oxopyrrolidinyl, 4-piperidonyl, pyrrolidinyl, hydantoinyl, valerolactamyl, oxiranyl, oxetanyl, tetrahydropyranyl, tetrahydrothiopyranyl, tetrahydropyrindinyl, tetrahydropyrimidinyl, tetrahydrothiopyranyl sulfone, tetrahydrothiopyranyl sulfoxide, morpholinyl, thiomorpholinyl, thiomorpholinyl sulfoxide, thiomorpholinyl sulfone, 1,3-dioxolane, tetrahydrofuranyl, dihydrofuranyl-2-one, tetrahydrothienyl, and tetrahydro-1,1-dioxothienyl. Typically, monocyclic heterocycloalkyl groups have 3 to 7 members. Preferred 3 to 7 membered monocyclic heterocycloalkyl groups are those having 5 or 6 ring atoms. A heteroatom may be substituted with a protecting group known to those of ordinary skill in the art, for example, the hydrogen on a nitrogen may be substituted with a tert-butoxycarbonyl group. Furthermore, heterocycloalkyl groups may be optionally substituted with one or more substituents. In addition, the point of attachment of a heterocyclic ring to another group may be at either a carbon atom or a heteroatom of a heterocyclic ring. Only stable isomers of such substituted heterocyclic groups are contemplated in this definition.

As used herein the term “substituent” or “substituted” means that a hydrogen radical on a compound or group is replaced with any desired group that is substantially stable to reaction conditions in an unprotected form or when protected using a protecting group. Examples of preferred substituents are those found in the exemplary compounds and embodiments disclosed herein, as well as halogen (chloro, iodo, bromo, or fluoro); alkyl; alkenyl; alkynyl; hydroxy; alkoxy; nitro; thiol; thioether; imine; cyano; amido; phosphonato; phosphine; carboxyl; thiocarbonyl; sulfonyl; sulfonamide; ketone; aldehyde; ester; oxygen (—O); haloalkyl (e.g., trifluoromethyl); cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl), monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); CO2CH3; CONH2; OCH2CONH2; NH2; SO2NH2; OCHF2; CF3; OCF3; and such moieties may also be optionally substituted by a fused-ring structure or bridge, for example —OCH2O—. These substituents may optionally be further substituted with a substituent selected from such groups. In certain embodiments, the term “substituent” or the adjective “substituted” refers to a substituent selected from the group consisting of an alkyl, an alkenyl, an alkynyl, an cycloalkyl, an cycloalkenyl, a heterocycloalkyl, an aryl, a heteroaryl, an aralkyl, a heteraralkyl, a haloalkyl, —C(O)NR11R12, —NR13C(O)R14, a halo, —OR13, cyano, nitro, a haloalkoxy, —C(O)R13, —NR11R12, —SR13, —C(O)OR13, —OC(O)R13, —NR13C(O)NR11R12, —OC(O)NR11R12, —NR13C(O)OR14, —S(O)ᵣR13, —NR13S(O)ᵣR14, —OS(O)ᵣR14, S(O)ᵣNR11R12, —O, —S, and —N—R13, wherein r is 1 or 2; R11 and R12, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R1 and R12 taken together with the nitrogen to which they are attached is optionally substituted heterocycloalkyl or optionally substituted heteroaryl; and R13.0 and R14 for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl. In certain embodiments, the term “substituent” or the adjective “substituted” refers to a solubilizing group.

The term “solubilizing group” means any group which can be substantially ionized and that enables the compound to be soluble in a desired solvent, such as, for example, water or water-containing solvent. Furthermore, the solubilizing group can be one that increases the compound or complex's lipophilicity. Typically, the solubilizing group is selected from alkyl group substituted with one or more heteroatoms such as N, O, S, each optionally substituted with alkyl group substituted independently with alkoxy, amino, alkylamino, dialkylamino, carboxyl, cyano, or substituted with cycloheteroalkyl or heteroaryl, or a phosphate, or a sulfate, or a carboxylic acid.

For example, by “solubilizing group” it is referred herein to one of the following:
an alkyl, cycloalkyl, aryl, heretoaryl group comprising either at least one nitrogen or oxygen heteroatom or which group is substituted by at least one amino group or oxo group.
an amino group which may be a saturated cyclic amino group which may be substituted by a group consisting of alkyl, alkoxycarbonyl, halogen, haloalkyl, hydroxyalkyl, amino, monoalkylamino, dialkylamino, carbamoyl, monoalkylcarbamoyl and dialkylcarbamoyl.
one of the structures a) to i) shown below, wherein the wavy line and the arrow line correspond to the point of attachment to core structure of formula I.

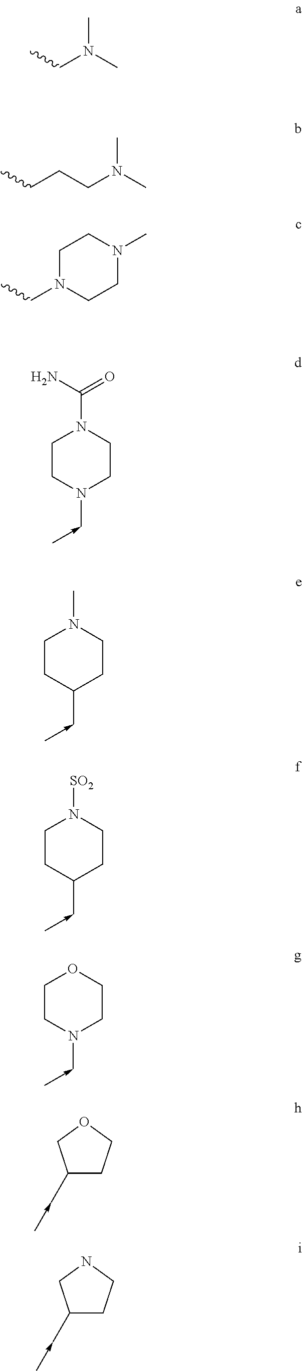

The term “cycloalkyl” means a saturated cyclic alkyl radical having from 3 to 10 carbon atoms. Representative cycloalkyls include cyclopropyl, 1-methylcyclopropyl, cyclobutyl, cyclopentyl, cyclohexyl, cycloheptyl, cyclooctyl, cyclononyl, and cyclodecyl. Cycloalkyl groups can be optionally substituted with one or more substituents.

The term “halogen” means —F, —Cl, —Br or —I.

In a particular embodiment the invention relates to a compound of formula II, or a pharmaceutical acceptable salt thereof,

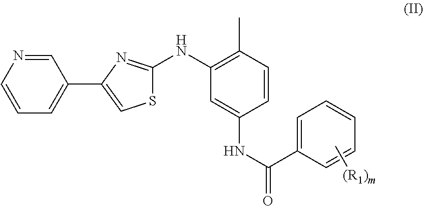

Wherein:

R1 is selected independently from hydrogen, halogen, a linear or branched alkyl, cycloalkyl group containing from 1 to 10 carbon atoms, trifluoromethyl, alkoxy, amino, alkylamino, dialkylamino, solubilizing group.
m is 0-5.

Masitinib is a c-Kit/FGFR3/PDGFR inhibitor with a potent anti-mast cell action.

We discovered in this regard, new potent and selective c-Kit, PDGFR and FGFR3 inhibitors which are 2-(3-aminoaryl)amino-4-aryl-thiazoles described in AB Science's PCT application WO 2004/014903.

Masitinib is a small molecule selectively inhibiting specific tyrosine kinases such as c-Kit, PDGFR, Lyn, Fyn and the fibroblast growth factor receptor 3 (FGFR3), without inhibiting, at therapeutic doses, kinases associated with known toxicities (i.e. those tyrosine kinases or tyrosine kinase receptors attributed to possible tyrosine kinase inhibitor cardiac toxicity, including ABL, KDR and Src) [Dubreuil et al., 2009, PLoS ONE 2009. 4(9):e7258]. The chemical name for masitinib is 4-(4-methylpiperazin-1-ylmethyl)-N-[4-methyl-3-(4-pyridin-3ylthiazol-2-ylamino) phenyl]benzamide—CAS number 790299-79-5, and the structure is shown below. Masitinib was first described in U.S. Pat. No. 7,423,055 and EP1525200B1. A detailed procedure for the synthesis of masitinib mesilate is given in WO2008/098949.

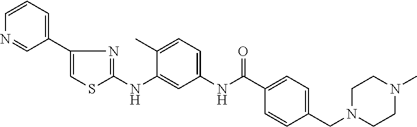 [SMILES: Cc1ccc(NC(=O)c2ccc(CN3CCN(C)CC3)cc2)cc1Nc1nc(-c2cccnc2)cs1]

Masitinib main kinase target is c-Kit, for which it has been shown to exert a strong inhibitory effect on wild-type and juxtamembrane-mutated c-Kit receptors, resulting in cell cycle arrest and apoptosis of cell lines dependent on c-Kit signaling [Dubreuil et al., 2009, PLoS ONE, 4(9):e7258]. Stem cell factor, the ligand of the c-Kit receptor, is a critical growth factor for mast cells; thus, masitinib is an effective anti-mastocyte, exerting a direct anti-proliferative and pro-apoptotic action on mast cells through its inhibition of c-Kit signaling. In vitro, masitinib demonstrated high activity and selectivity against c-Kit, inhibiting recombinant human wild-type c-Kit with an half inhibitory concentration (IC₅₀) of 200±40 nM and blocking stem cell factor-induced proliferation and c-Kit tyrosine phosphorylation with an IC₅₀ of 150±80 nM in Ba/F3 cells expressing human or mouse wild-type c-Kit. In addition to its anti-proliferative properties, masitinib can also regulate the activation of mast cells through its targeting of Lyn and Fyn, key components of the transduction pathway leading to IgE induced degranulation [Gilfillan & Tkaczyk, 2006, Nat Rev Immunol, 6:218-230] [Gilfillan et al., 2009, Immunological Reviews, 228:149-169]. This can be observed in the inhibition of FcεRI-mediated degranulation of human cord blood mast cells [Dubreuil et al., 2009, PLoS ONE; 4(9):e7258]. It has been shown in vitro that masitinib has activity against the FGFR3 kinase at concentrations that are attainable in vivo (IC₅₀ 1.5-2 μM) (See Example #1). Masitinib is also a potent inhibitor of PDGFR α and β receptors. Recombinant assays show that masitinib inhibits the in vitro protein kinase activity of PDGFR-α and β with IC₅₀ values of 540±60 nM and 800±120 nM. In Ba/F3 cells expressing PDGFR-α, masitinib inhibited PDGF-BB-stimulated proliferation and PDGFR-α tyrosine phosphorylation with an IC₅₀ of 300±5 nM.

Treatment of Multiple Myeloma with Masitinib

In connection with the present invention, our preclinical data support anti-tumoral properties of masitinib in multiple myeloma (see Example #1). Masitinib processes a unique dual inhibitory activity on c-Kit and FGFR3, properties which are not found in any other tyrosine kinase inhibitors. We have discovered that masitinib inhibits FGFR3 phosphorylation and demonstrates an anti-proliferative activity in fresh plasma cells from patients with t(4;14) multiple myeloma expressing FGFR3. Both t(4;14) positive patient-derived plasma cells and cell lines were sensitive to masitinib for cell growth and survival. Furthermore, synergy was observed in combination with dexamethasone, which is in line with findings from Montero et al (2008, hematological 93(6):851-8) that inhibition of c-Kit may act in a synergistic manner with anti-myeloma agents. These preclinical data also showed that masitinib exerts an inhibitory action against t(4;14)-negative multiple myeloma cell lines, indicating that masitinib may also be of therapeutic potential to multiple myeloma patients who do not bear the t(4:14) translocation.

Our preclinical data on masitinib's kinase inhibition profile is in line with the possibility that masitinib exerts anti-angiogenesis properties in multiple myeloma via inhibition of PDGFR-beta [Dubreuil et al., 2009, PLoS ONE; 4(9):e7258]. Moreover, PDGFR, c-Kit, and FGFR3 kinases have been shown to promote multiple myeloma cell resistance to anti-myeloma agents, including bortezomib, dexamethasone, and melphalan. In addition, mast cells contain a number of cytokines (e.g. IL-6) that when released into the bone marrow milieu can contribute to the development of drug resistance in multiple myeloma. Thus, masitinib's inhibitory action can be expected to alleviate the development of drug resistance when administered in combination with relevant anti-myeloma agents.

Considering the synergistic effects of masitinib on different pathways involved in t(4;14) multiple myeloma, we investigated the efficacy and safety of oral masitinib in patients suffering from relapsed or refractory t(4;14) multiple myeloma. We also tested if masitinib could be of benefit independent of FGFR3 expression and regardless of the t(4;14) translocation. Evidence that masitinib is a viable therapeutic strategy for treatment of multiple myeloma was demonstrated through positive response according to measures of cancer survival in two phase 2 studies (see Examples #2 and 3). In the first of these clinical trials, masitinib was administered orally in monotherapy until disease progression, after which dexamethasone was added in combination. In the second clinical trial, masitinib was administered orally in combination with bortezomib associated to dexamethasone. The main conclusions from these studies were as follows:
Masitinib monotherapy had an acceptable safety profile but showed limited efficacy in patients with refractory or relapsed multiple myeloma with the t(4,14) translocation.
Masitinib combination with dexamethasone had an acceptable safety profile and showed efficacy in patients with refractory or relapsed multiple myeloma with the t(4,14) translocation, irrespective of FGFR3 expression status.
Masitinib combination with bortezomib and dexamethasone had an acceptable safety profile and showed efficacy in patients with refractory or relapsed multiple myeloma irrespective of the t(4,14) translocation, i.e. t(4,14)-positive and t(4,14)-negative patients.

In connection with the present invention, it would seem, without wishing to be bound by the theory that surprisingly a tyrosine kinase inhibitor or a mast cell inhibitor, notably as defined above, especially masitinib, could also be of further therapeutic benefit against multiple myeloma by inhibiting mast cell proliferation and degranulation via inhibition of c-Kit, Lyn and Fyn. This is significant as it represents a mechanism of action that is independent from chromosomal abnormalities of myeloma cells and could therefore be effective across a broad range of multiple myeloma subtypes. It follows that the masitinib-induced decrease in overall mast cell burden and mast cell degranulation would lead to a reduction of mast cell mediator release, including, but not limited to, pro-proliferation cytokines of myeloma cells such as interleukin IL-6 and TNF, and pro-angiogenic mediators such as IL-8, VEGF, and Ang-1. In addition, a reduction in release of various chemoattractants associated with mast cell migration will lessen the rate of mast cell recruitment and accumulation, further inhibiting these processes.

Thus, a tyrosine kinase inhibitor or a mast cell inhibitor, notably as defined above, especially masitinib with its anti-mast cell properties and combined c-Kit/FGFR3/PDGFR inhibition, is well adapted to the treatment of multiple myeloma, and in particular patients with t(4;14) multiple myeloma. Masitinib's effect is multifactorial and may include inhibition of myeloma cell proliferation, induction of myeloma cell apoptosis, reduced drug resistance, synergistic action with conventional anti-myeloma agents, and inhibition of angiogenesis. When administered in association with an additional care in multiple myeloma (for example, initially in combination with at least one other targeted therapy, alkylating agent, corticosteroid, or immunomodulatory agent), this strategy may result in disease remission followed by the induction and maintenance of a period of tumor control. This would allow for improved safety of conventional anti-myeloma agents with the possibility for prolonged uninterrupted treatment periods at lower doses thereby, preventing development of drug resistance or even overcoming drug resistance. Unexpectedly, without wishing to be bound by the theory, it is through this multifaceted mechanism of action that a compound of the invention (i.e. a tyrosine kinase inhibitor or a mast cell inhibitor, especially masitinib or a pharmaceutically acceptable salt thereof), can elicit a response in multiple myeloma patients.

Thus, in a first embodiment, the invention relates to the use of at least one compound of the invention for the preparation of a medicament for the treatment of multiple myeloma, especially for the treatment of refractory or first relapsed multiple myeloma, and in particular patients with t(4;14) multiple myeloma, comprising administration of a tyrosine kinase inhibitor or a mast cell inhibitor, especially masitinib or a pharmaceutically acceptable salt thereof, administered in association with an additional care in multiple myeloma; for example, autologous stem-cell transplantation, targeted therapies, anti-myeloma agents such as alkylating agents, corticosteroids, or immunomodulatory agents, including bortezomib, lenalidomide, and dexamethasone.

The invention thus relates to a method of treatment of multiple myeloma, especially for the treatment of refractory or first relapsed multiple myeloma, and in particular patients with t(4;14) multiple myeloma, wherein a tyrosine kinase inhibitor or a mast cell inhibitor, especially masitinib or a pharmaceutically acceptable salt thereof, administered in association with an additional care in multiple myeloma; for example, autologous stem-cell transplantation, targeted therapies, anti-myeloma agents such as alkylating agents, corticosteroids, or immunomodulatory agents, including bortezomib, lenalidomide, and dexamethasone.

Advantageously, said patients are those afflicted by relapsing multiple myeloma, as defined by the International uniform response criteria for multiple myeloma (International Myeloma Working Group criteria) who received one previous therapy.

According to another embodiment, said patients are those afflicted by refractory multiple myeloma, including patients resistant to bortezomib, lenalidomide, and/or dexamethasone.

According to a preferred embodiment, said patients are those afflicted by t(4;14) multiple myeloma with expression of FGFR3.

According to another preferred embodiment, said patients are those afflicted by multiple myeloma expressing c-Kit, especially the c-Kit GNNK-negative form.

A patient according to the invention is in particular a human.

A preferred salt of masitinib is masitinib mesilate.

According to one embodiment, a compound of the invention is an inhibitor of wild-type c-Kit, PDGFR, Lyn and Fyn kinase activity.

According to another embodiment, a compound of the invention is a dual c-Kit/FGFR3 inhibitor.

According to another embodiment, a compound of the invention is to be administered at a starting daily dose of 3.0 mg/kg/day to 9.0 mg/kg/day, with the preferred embodiment for patients with refractory or first relapsed multiple myeloma being a starting daily dose of 6.0 mg/kg/day ±1.5 mg/kg/day.

Preferably, a compound of the invention is dose escalated by increments of 1.5 mg/kg/day to reach a maximum of 12.0 mg/kg/day, notably in low responder patients.

Said compound of the invention is preferably administered orally.

Said compound of the invention is preferably administered twice a day.

Indeed, depending on age, individual condition, mode of administration, and the clinical setting, effective doses of said tyrosine kinase inhibitor or mast cell inhibitor, especially masitinib or a pharmaceutically acceptable salt thereof, in human patients with multiple myeloma are 3.0 mg/kg/day to 9.0 mg/kg/day per os, preferably in two daily intakes. For adult human patients with multiple myeloma, and in particular patients with refractory or first relapsed multiple myeloma, and especially patients with t(4;14) multiple myeloma, a starting dose of said tyrosine kinase inhibitor or mast cell inhibitor, especially masitinib or a pharmaceutically acceptable salt thereof, of 6.0 mg/kg/day ±1.5 mg/kg/day has been found to be the preferred embodiment according to the invention. For patients with an inadequate response after an assessment of response to therapy and in the absence of limiting toxicities, dose escalation of said tyrosine kinase inhibitor or mast cell inhibitor, especially masitinib or a pharmaceutically acceptable salt thereof, to a maximum of 12.0 mg/kg/day can be safely considered and patients may be treated as long as they benefit from treatment and in the absence of limiting toxicities.

Dose adjustment can be considered a dynamic process, with a patient undergoing multiple increases and/or decreases to optimize the balance between response and toxicity throughout treatment, both of which are likely to vary over time and duration of drug exposure. If dose escalation is undertaken, it is suggested that the starting dose of 6.0±1.5 mg/kg/day be incremented by 1 to 2 mg/kg/day up to a maximum dose of 12.0 mg/kg/day, over a period which depends upon clinical observations. For example, a single dose escalation of said tyrosine kinase inhibitor or mast cell inhibitor, especially masitinib or a pharmaceutically acceptable salt thereof, and preferably masitinib mesilate may take from 1 to 2 months. It is also contemplated herein that to fully obtain the therapeutic benefits of a patient-optimized dose of said tyrosine kinase inhibitor or mast cell inhibitor, especially masitinib or a pharmaceutically acceptable salt thereof, dose increments smaller than 1 to 2 mg/kg/day could be implemented. Dose reduction is to be considered to reduce toxicity in appropriate cases.

Any dose indicated herein refers to the amount of active ingredient as such, not to its salt form.

For example, said pharmaceutical composition comprises a dose of at least 50 mg and less than 150 mg, and preferably of 100 mg, of said compound(s) of the invention.

For example, said pharmaceutical composition comprises a dose of at least 150 mg and less than 400 mg, and preferably of 200 mg, of said compound(s) of the invention.

According to a preferred embodiment, the compound of the invention is administered for the treatment of multiple myeloma, especially for the treatment of refractory or first relapsed multiple myeloma, and in particular patients with t(4;14) multiple myeloma, in combination with at least one other targeted therapy agent, alkylating agent, corticosteroid, or immunomodulatory agent.

The additional care or second medication is preferably administering one compound or agent selected from the group consisting of: bortezomib, dexamethasone, thalidomide, lenalidomide, doxorubicin, vincristine, melphalan, cyclophosphamide, pomalidomide, carfilzomib, elotuzumab, vorinostat, and panabinostat; and any combination of these drugs.

The compound(s) of the invention and one or more targeted therapy agent, alkylating agent, corticosteroid, or immunomodulatory agent, may be administered separately, simultaneously or sequentially in time.

The use or method comprises a long-term administration of an effective amount of said tyrosine kinase inhibitor or mast cell inhibitor, especially masitinib or a pharmaceutically acceptable salt thereof, over more than 3 months, preferably more than 12 months.

In yet another embodiment, the invention also relates to a method of treatment of multiple myeloma, wherein a tyrosine kinase inhibitor or a mast cell inhibitor, especially masitinib or a pharmaceutically acceptable salt thereof, acts as an adjuvant or maintenance therapy to prevent relapse following treatment-induced remission or post-autologous stem-cell transplantation, optionally combined with at least one other anti-myeloma agent.

The invention also relates to a tyrosine kinase inhibitor or a mast cell inhibitor, notably as defined above, especially masitinib for use as a medicament or in a pharmaceutical composition for a method as defined in the description.

In another embodiment, the invention also relates to a method of treatment of multiple myeloma, especially for the treatment of refractory or first relapsed multiple myeloma, and in particular patients with t(4;14) multiple myeloma, wherein a tyrosine kinase inhibitor or a mast cell inhibitor, especially masitinib or a pharmaceutically acceptable salt thereof, is administered for the treatment of multiple myeloma in combination with at least one other targeted therapy agent, alkylating agent, corticosteroid, or immunomodulatory agent; for example, bortezomib, dexamethasone, thalidomide, lenalidomide, doxorubicin, vincristine, melphalan, cyclophosphamide, pomalidomide, carfilzomib, elotuzumab, vorinostat, or panabinostat.

In one embodiment, said tyrosine kinase inhibitor or mast cell inhibitor, especially masitinib or a pharmaceutically acceptable salt thereof, is administered for the treatment of multiple myeloma, wherein said patients have relapsing multiple myeloma, as defined by the International uniform response criteria for multiple myeloma (International Myeloma Working Group criteria), who received one previous therapy

According to an embodiment, said tyrosine kinase inhibitor or mast cell inhibitor, especially masitinib or a pharmaceutically acceptable salt thereof, is administered for the treatment of multiple myeloma, wherein said patients have refractory multiple myeloma, including patients resistant to bortezomib, lenalidomide, and/or dexamethasone.

According to another embodiment, said tyrosine kinase inhibitor or mast cell inhibitor, especially masitinib or a pharmaceutically acceptable salt thereof, is administered for the treatment of multiple myeloma, wherein said patients have t(4;14)-positive multiple myeloma, and in particular t(4;14) multiple myeloma with FGFR3 expression.

According to another embodiment, said tyrosine kinase inhibitor or mast cell inhibitor, especially masitinib or a pharmaceutically acceptable salt thereof, is administered for the treatment of multiple myeloma, wherein said patients have t(4;14)-negative multiple myeloma.

According to another embodiment, said tyrosine kinase inhibitor or mast cell inhibitor, especially masitinib or a pharmaceutically acceptable salt thereof, is administered for the treatment of multiple myeloma, wherein said patients have multiple myeloma expressing c-Kit, especially the c-Kit GNNK-negative form

Pharmaceutically acceptable salts are pharmaceutically acceptable acid addition salts, like for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic, in particular methanesulfonic acid (or mesilate), or aromatic sulfonic acids, for example benzene-, p-toluene- or naphthalene-2-sulfonic acid.

In a preferred embodiment of the above-depicted treatment, the active ingredient masitinib is administered in the form of masitinib mesilate; which is the orally bioavailable mesylate salt of masitinib—CAS 1048007-93-7 (MsOH); C28H30N6OS.CH3SO3H; MW 594.76:

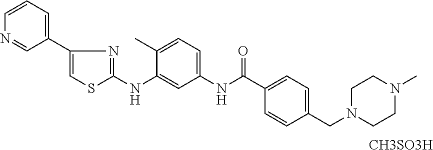

Given that the masitinib dose in mg/kg/day used in the described dose regimens refers to the amount of active ingredient masitinib, compositional variations of a pharmaceutically acceptable salt of masitinib mesilate will not change the said dose regimens.

Masitinib may be administered via different routes of administration but oral administration is preferred. Thus, in still another preferred embodiment, in the use or the method above, masitinib or salts thereof, is administered orally; preferably twice a day for long term period such as over more than 3 months, preferably more than 12 months. Masitinib can be administered in the form of 100 and 200 mg tablets.

According to a particular embodiment, the composition of the invention is an oral composition.

As is known to the person skilled in the art, various forms of excipients can be used adapted to the mode of administration and some of them can promote the effectiveness of the active molecule, e.g. by promoting a release profile rendering this active molecule overall more effective for the treatment desired.

The pharmaceutical compositions of the invention are thus able to be administered in various forms, more specially for example in an injectable, pulverizable or ingestible form, for example via the intramuscular, intravenous, subcutaneous, intradermal, oral, topical, rectal, vaginal, ophthalmic, nasal, transdermal or parenteral route. A preferred route is oral administration. The present invention notably covers the use of a compound according to the present invention for the manufacture of pharmaceutical composition.

Such medicament can take the form of a pharmaceutical composition adapted for oral administration, which can be formulated using pharmaceutically acceptable carriers well known in the art in suitable dosages. Such carriers enable the pharmaceutical compositions to be formulated as tablets, pills, dragees, capsules, liquids, gels, syrups, slurries, suspensions, and the like, for ingestion by the patient. In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.). The present inventions also covers a single pharmaceutical packaging comprising a tyrosine kinase inhibitor or a mast cell inhibitor, especially masitinib or a pharmaceutically acceptable salt thereof and at least one anti-myeloma agent, including notably alkylating agents, corticosteroids, or immunomodulatory agents, including notably bortezomib, dexamethasone, thalidomide, lenalidomide, doxorubicin, vincristine, melphalan, cyclophosphamide, pomalidomide, carfilzomib, elotuzumab, vorinostat, and panabinostat.

The present invention is illustrated by means of the following examples.

### Example 1

### Pre-Clinical Studies

### Methods and Materials

### Cells:

Multiple myeloma cell lines RPMI 8226, U266 (t(4;14) negative) and OPM2, LP1, NCI-H929 (t(4;14) positive) were cultured in RPMI medium (Gibco-BRL) supplemented with 10% fetal calf serum (FCS, Life Technologies), 100 units/ml penicillin, 100 μg/ml streptomycin, except LP1 which were cultured in DMEM (Gibco-BRL). Bone marrow plasma cells were CD138-selected using MACS systems (Miltenyi Biotec, Germany).

### Proliferation Assays:

Cells were incubated in the presence of masitinib and/or dexamethasone as indicated for 48 hours and 0.5×10⁴ cells/well were seeded in 96-well microtiter plates. [6-³H] thymidine (Amersham Biosciences, UK) was added (1 μCi/well) for 16 hours. Thymidine incorporation was measured by liquid scintillation counting (Wallace, PerkinElmer). All experiments were performed in triplicate.

### Expression of Human FGFR in Ba/F3 Cells:

The cDNA encoding the human wild-type FGFR3 was subcloned in the LXSN retroviral vector (Takara Bio Europe/Clontech, France), generating plasmid LfgfWTSN. The constructs cloned in the LXSN vector were used to transfect the ecotropic packaging cell line GP+E-86 for virus production. Ba/F3 cells were then infected by co-culture for 24-48 hours with transfected GP+E-86 cells that had been pretreated for 3 hours with 10 μg/ml mitomycin C.

### Analysis of Tyrosine Phosphorylation in Ba/F3 Cells:

Masitinib was dissolved as a 10 or 20 mM stock solution in dimethylsulfoxide and stored at −80° C. For each experiment fresh dilutions were prepared. Ba/F3 cells (5×10⁶) expressing FGFR3 were serum-starved for 1-3 hours. Cells expressing FGFR3 were stimulated with 10 ng/ml FGF (Invitrogen). The cells were then placed on ice and washed with ice-cold phosphate-buffered saline, lysed in 500 μl of ice-cold HNTG buffer (50 mM HEPES pH 7, 50 mM NaF, 1 mM EGTA, 150 mM NaCl, 1% Triton X-100, 10% glycerol, and 1.5 mM MgCl₂) containing a mixture of protease inhibitors (Roche Applied Science, Meylan, France) and 100 μM Na₃VO₄. Lysates were separated by sodium dodecyl sulfate-polyacrylamide gel electrophoresis on an 8% acrylamide gel. Proteins were electrophoretically transferred onto a polyvinylidene difluoride membrane (Millipore SAS, St. Quentin en Yvelines, France). Membranes were blocked in Tris-buffered saline containing 0.05% Tween-20 and 5% bovine serum albumin for 30 to 45 minutes at room temperature. Western blotting was performed using 1:1000 anti-phosphotyrosine antibody 4G10 (Cell Signaling Technology), followed by 1:20,000 horseradish peroxidase-conjugated anti-mouse antibody. Immunoreactive bands were detected using enhanced chemiluminescent reagents (Pierce, Rockford, Ill., USA).

### Results

### Effect of Masitinib on FGFR3 Phosphorylation and on Proliferation of FGFR3 Expressing Ba/F3 Cells:

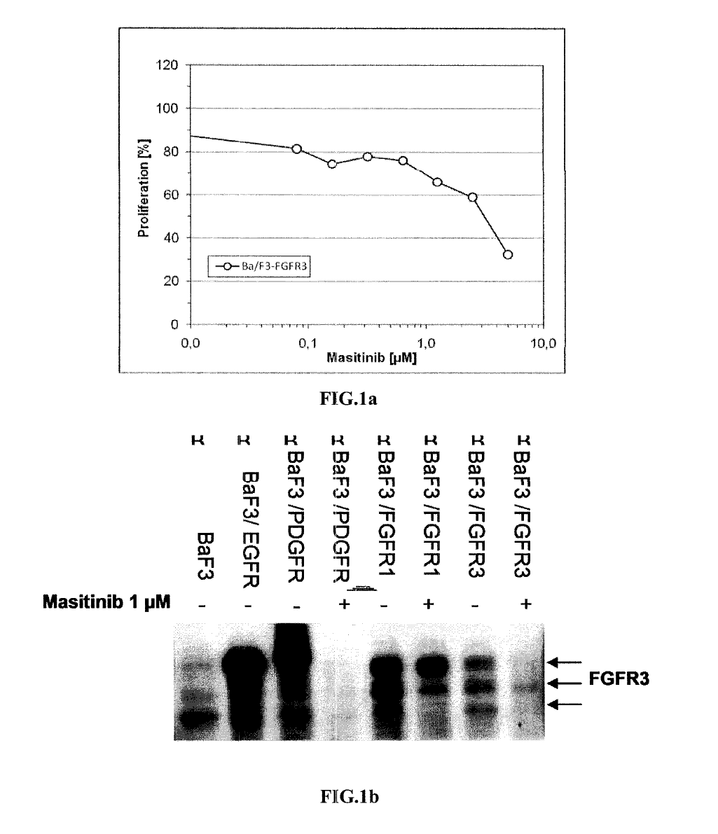

As shown in FIG. 1A, masitinib inhibited the growth of BaF3 cell expressing the intracellular kinase domain of FGFR3. In addition the phosphorylation of the intracellular domain of FGFR3 but not of FGFR1 was inhibited in the presence of masitinib (1 μM). Inhibition of the PDGF receptor is shown as a positive control (FIG. 1B).

### FIG. 1: Effect of Masitinib on Proliferation of FGFR3 Expressing Ba/F3 Cell and on FGFR3 Phosphorylation

(A) Masitinib dose-dependent inhibition of Ba/F3 cell proliferation expressing FGFR3. (B) Western analysis of the phosphorylation pattern following stimulation of transfected Ba/F3 cells with 10 ng/ml FGF. The phosphorylation of the intracellular domain of FGFR3, but not of FGFR1, was inhibited in the presence of masitinib (1 μM) Inhibition of the phosphorylation of the intrinsic PDGF receptor is shown as a positive control.

Effect of Masitinib on Multiple Myeloma Cell Lines and Plasma Cells from Patients:

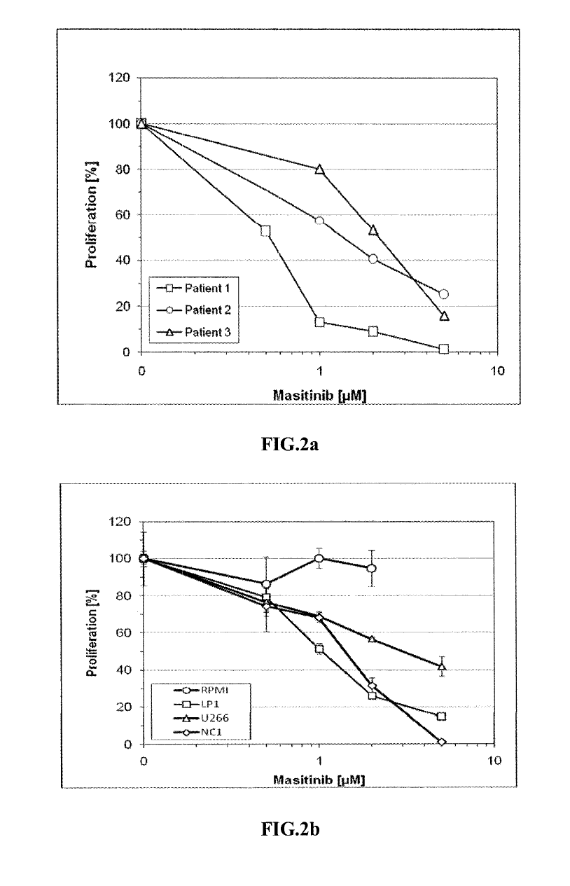

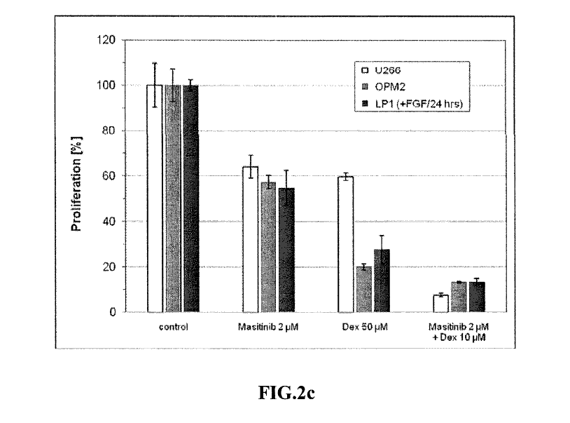

The proliferation of t(4;14) cell lines LP1 and NCI-H929 was inhibited by 50% in the presence of masitinib (2 μM), in a dose-dependent manner. Masitinib had no effect on the t(4;14) negative cell line RPMI, while the proliferation of another t(4;14) negative cell line U266 was significantly and dose-dependently decreased. Masitinib also inhibited the proliferation of fresh plasma cells from patients with t(4;14) multiple myeloma expressing FGFR3. Anti-proliferative synergy with dexamethasone was observed in the following cell lines: U266 (t(4;14) negative), OPM2 (t(4;14) positive), and FGF-activated LP1 cells (t(4;14) positive) (see FIG. 2). When compared to the effect of dexamethasone alone, the addition of masitinib significantly reduced proliferation 2-fold (OPM2 and LP1) or 7-fold (U266) obtained with 5 times less dexamethasone.

FIG. 2: In Vitro Proliferation Experiments with Cells Derived from Patients and Myeloma Cell Lines with Masitinib Alone or in Combination with Dexamethasone.

(A) Plasma cells of three multiple myeloma patients (one enrolled in the study). (B) The cell line NCl carries the translocation t(4;14), while the lines RPMI and U266 (cell lines which ectopically expresses FGFR3) do not. The line LP1 is characterized by the translocation and additional expression of FGFR3. (C) Anti-proliferative synergy in cell lines U266, OPM2 and LP1 with the combination of masitinib plus dexamethasone. While the line OPM2 has constitutively activated FGFR3 due to the mutation K650E in the kinase domain, LP1 cells were stimulated with FGF.

These in-vitro data show that masitinib can inhibit FGFR3 phosphorylation, proliferation of multiple myeloma cell lines, both positive and negative t(4:14), and patient-derived plasma cells, with synergy observed when combined with dexamethasone. Together this provides a compelling rationale to investigate the effect of masitinib in patients with positive or negative t(4;14) multiple myeloma.

### Example 2

### Clinical Evaluation of Masitinib Plus Dexamethasone in Patients with Multiple Myeloma

### Methods

This open-label phase 2 study investigated the safety and efficacy of masitinib in patients with relapsed or refractory t(4;14) multiple myeloma regardless of FGFR3 expression. The main objective was to provide the first evidence of therapeutic effect of masitinib in the treatment of patients suffering from multiple myeloma with t(4;14) translocation. Masitinib monotherapy (9 mg/kg/day) was administered orally until disease progression, after which dexamethasone (40 mg/day, 4 days/month) was added in combination. Included were patients with t(4;14) multiple myeloma in first relapse (second line) or beyond (third, fourth line etc). All patients had detectable and quantifiable monoclonal M-component, a life expectancy of more than 3 months, and ECOG 0-2. Confirmation of multiple myeloma diagnosis was made with detection of the t(4;14) translocation by FISH and PCR, with FGFR3 expression identified by FACS. End-points were best response rate and progression free survival (PFS), using the revised Blade criteria (International Myeloma Working Group, IMWG) [Durie B G, et al. Leukemia 20:7, 2006].

### Results

### Baseline

Between February 2005 and September 2006, 24 multiple myeloma patients enrolled at eight centers across France. All 24 patients had translocation t(4;14), and 21 patients expressed FGFR3 (Table 2). Six patients were in first relapse with non-aggressive relapse and two patients enrolled at plateau phase after a first line of treatment (i.e. unsatisfactory treatment response) with partial response and >75% decrease of monoclonal immunoglobulin. Sixteen patients were in second or more relapses (from 3 to 6 lines), including nine patients with non-aggressive multiple myeloma. For the seven patients in aggressive relapse the following treatment regimens were initiated prior to commencement of masitinib therapy: bortezomib/thalidomide/dexamethasone (N=5); thalidomide/dexamethasone (N=1); and high dose melphalan (N=1).

TABLE 2
Multiple myeloma disease characteristics, ITT population

| Parameter | N = 24 |
|---|---|
| Female N (%) | 16 (66.7%) |
| Age (years) | |
| Mean ± Std | 61.1 ± 10.1 |
| Min-Max | 44.0-83.0 |
| Time since diagnosis (years) | |
| Mean ± Std | 4 ± 3 |
| Median | 3 |
| Min-Max | 0-12 |
| Previous Treatment | |
| First relapse | 6 (25.0%) |
| Beyond first relapse | 16 (66.7%) |
| Unsatisfactory response | 2 (8.3%) |
| Relapse in patients with >2 prior lines | |
| (N = 16) | |
| Aggressive relapse | 7 (43.8%) |
| Non aggressive relapse | 9 (56.3%) |
| Translocation t(4, 14) | |
| + | 24 (100.0%) |
| FGFR3 expression | |
| − | 3 (12.5%) |
| + | 21 (87.5%) |

### Efficacy Analysis

Masitinib monotherapy induced no response in the study population. At the end of the monotherapy treatment phase a total of seventeen patients entered the combination therapy masitinib plus dexamethasone, however, one patient could not be evaluated because of early progression. Of the seven patients not entering the combination treatment phase, two patients had died due to rapid disease progression and five patients withdrew due to treatment-related adverse events, of which three were grade 3 (renal failure, nausea/vomiting, and rash).

Under combination therapy, one patient recorded a very good partial response (IMWG criteria), this patient was in second line of treatment (i.e. first relapse) with non-aggressive relapse, and 5 patients recorded Minor Response. No response was observed in patients with aggressive relapse.

The median progression free survival (PFS) was 3.9 months in all 24 patients, and for subgroups:
9.1 months for patients in their second line of treatment (i.e. first relapse),
3.0 months for patients that received more than two previous lines of treatment (i.e. beyond first relapse); with sub-analysis according to non-aggressive and aggressive relapse being 3.9 and 2.1 months, respectively.

The median overall survival was 19.8 months in all 24 patients, and for subgroups:
35.7 months for patients in their second line of treatment (i.e. first relapse),
8.6 months for patients that received more than two previous lines of treatment (i.e. beyond first relapse); with sub-analysis according to non-aggressive and aggressive relapse being 25.4 and 8.1 months, respectively.

Overall, 43.5% of the patients were still alive after 2 years. All patients with unsatisfactory response to previous treatment or in first relapse survived for at least 1 year.

### Safety Analysis

Twenty-four patients received at least one dose of study medication. A total of 145 suspected masitinib-related (or not assessable) adverse events (AE) were reported in 21 patients (87.5%). Seven patients (29.2%) experienced at least one suspected masitinib-related (or not assessable) serious adverse event (SAE). No death was suspected to be due to masitinib. Five patients (20.8%) discontinued the study because of AEs suspected to be masitinib-related.

The most frequent suspected (or not assessable) AEs were nausea/and or vomiting, recorded for 16 patients (66.7%), suspected diarrhea was reported in 10 patients (41.7%), and rashes in 2 patients (8.3%). Drug-related edema was observed in 13 (54.2%) patients. Severe suspected (or not assessable) adverse events, defined as non-hematological grade 3/4 events, were reported in seven patients (29.2%). Three patients presented with severe nausea and two with acute renal failure. Severe edema was reported in two patients but no other severe suspected (or not assessable) adverse events were reported more than once per patient.

### Discussion and Conclusion

In this study the combination of dexamethasone with masitinib was demonstrated to be safe and encouraging efficacy on PFS and overall survival in the global population of patients with multiple myeloma t(4;14) was observed. Therapeutic benefit, as evidenced by median PFS and overall survival, is especially apparent in patients of the second line treatment (first relapse) subpopulation, and in patients beyond second line treatment with non-aggressive relapse. The study strategy to test sequentially masitinib monotherapy followed by combined treatment with dexamethasone has shown some promising results but is possibly not aggressive enough for those patients with the worst prognostic factors, i.e. the subpopulation of patients beyond second line treatment with aggressive relapse.

In this phase 2 study the median PFS of patients in first relapse (N=6) reached 9.1 months. This is twice the median PFS of 4.7 months reported in a study of ten patients with t(4;14) multiple myeloma relapsing after high-dose therapy with autologous stem cell transplantation (ASCT), treated with thalidomide plus dexamethasone [Jaksic et al. J Clin Oncol 23:7069-73, 2005]. Furthermore, median overall survival for patients in second line treatment (first relapse) was 35.7 months for masitinib as compared to 10 months in the Jaksic study. This shows that the combination of masitinib with dexamethasone offers an improvement over current therapy in multiple myeloma patients with translocation t(4;14), as well as a comparable safety profile. It is also noted that current study's efficacy results are likely to be underestimated due to transient interruptions of masitinib treatment. For example, six patients (25%) were treated for less than 30 days during the first 2 months. These early masitinib transient interruptions and discontinuations were observed mainly in patients with severe aggressive relapse at entry or in patients who had been discontinued prematurely due to early onset of AEs. Eight patients dropped out of the study before the introduction of the combined treatment masitinib plus dexamethasone.

### Example 3

### Clinical Evaluation of Masitinib Plus Bortezomib Associated to Dexamethasone in Patients with Relapsing or Refractory Multiple Myeloma

### Methods

This open-label phase 2 study investigated the safety and efficacy of masitinib administered in combination with other chemotherapy regimens, including masitinib plus bortezomib (Velcade) in association with dexamethasone, in patients with relapsing or refractory multiple myeloma. Initially, this study was to investigate the safety and efficacy of masitinib administered in combination with the chemotherapy regimens of masitinib plus (dexamethasone/bortezomib/thalidomide); or masitinib plus (dexamethasone/bortezomib/adriamycin). However, after early preliminary results both these latter arms were eliminated from the study due to a tendency of higher incidence of adverse events or because these were not considered a gold standard treatment in this pathology.

Drugs were administered at the following levels:

Masitinib: 9 mg/kg/day, orally, twice daily.
Velcade: 1.3 mg/m² intravenously; Level 1: Days 1 and 4; Level 2: Days 1, 4, 8 and 11
Dexamethasone: 40 mg/day, orally; Level 1: Days 1 to 4; Level 2: Days 1 to 4, 8 and 11
Adriamycin: 9 mg/m2 intravenously (bolus or perfusion); Days 1 to 4
Thalidomide: 200 mg/day, orally, once daily in the evening

For the masitinib plus bortezomib (Velcade) in association with dexamethasone combination two cohorts at different dose levels (according to frequency of administration over a given 21 day cycle) were tested with masitinib administered at 9 mg/kg/day, orally, twice daily, in combination with 4 to 8 cycles of bortezomib plus dexamethasone (one cycle was 21 days). The two dosing regimens of the bortezomib plus dexamethasone combination (VD) were successively tested. After the observation that a low level of VD was well tolerated in four patients, the following patients were treated at a higher level. Included were patients with refractory or relapsing multiple myeloma:
With t(4; 14) translocation regardless of FGFR3 status, i.e. expressing FGFR3 or not;
or without a t(4; 14) translocation and relapsing within 1 year after the first line therapy including or not bone marrow transplantation.

All patients were to receive combination therapy for the planned number of cycles or until disease progression, after which they could continue with masitinib plus dexamethasone in an extension phase. In the case of response and stable disease, masitinib and dexamethasone were to be continued until disease progression at the same dosage as the previous cycles. All patients had detectable and quantifiable monoclonal M-component, a life expectancy of more than 3 months, adequate organ function, and ECOG 0-2.

### Results

Only those findings from the masitinib plus bortezomib/dexamethasone combination (VD) will be reported hereafter. Overall, seventeen patients were recruited (4 patients in VD level 1 and 13 patients in VD level 2). All received at least one dose of treatment and 14 patients were treated for at least 1 month. Eleven patients were treated for more than 3 months. 71% of patients experienced at least one AE suspected to be treatment-related and 12% of patients experienced at least one grade 4 AE suspected to be treatment-related.

In cohort VD Level 1, all four patients enrolled received at least one dose of treatment. Three out of the four patients were treated for more than 1 month. All patients experienced at least one AE that was suspected to be treatment-related. No grade 4 adverse event suspected to be treatment-related was reported. In cohort VD Level 2, all 13 patients received at least one dose of treatment, of which 8 (62%) experienced at least one AE that was suspected to be treatment-related. Of the 13 patients enrolled, two patients (15%) experienced at least one grade 4 AE suspected to be treatment-related and two patients (15%) experienced a grade 3 AE suspected to be treatment-related. In this cohort, nine patients were treated for at least 3 months.

Median Progression free survival (PFS) was 7.4 months (range 0-21.6) in the overall VD population. PFS was longer in the 13 patients treated at level 2 (7.4 months, range 0.4-21.6) compared to the 4 patients treated at level 1 (2.9 months, range 0-10.5). Median PFS in the 13 patients receiving VD as second line therapy (i.e. first relapse) was 9.3 months (range 0-21.6). Median PFS according to t(14;4) status was 9.3 months (range 0-16.7) in t(4;14) negative patients and 5.9 months (range 0,4-21,6) in t(4;14) positive patients.

Five of the 17 VD patients (29%) were still alive at the cut-off date of 31 Mar. 2010. Median overall survival (OS) was 18.1 months (range 0.4-28.9) in all patients, and was 20.0 months in 13 patients treated at level 2 (range 0.4-28.3). Median OS was 22.3 months (range 4.2-28.9) in the 13 patients receiving VD as second line therapy (i.e. first relapse). Median OS according to t(14;4) status was 25.3 months (range 4.2-28.3) in t(4;14) negative patients and 17.1 months (range 0.4-28.9) in t(4;14) positive patients.

A response rate (complete response or partial response) of 75% was reported in the intent-to-treat population, including five patients with a very good partial response, and was 86% in the per-protocol population. Response according to t(14;4) status was 8 out of 10 patients (80%) reporting a best response of partial response in t(4;14) positive patients compared to three of the five patients (60%) in t(4;14) negative patients. Eleven of the 12 patients (92%) receiving VD as second line therapy had a best response of partial response.

### Conclusion

This study revealed that masitinib can be used in combination with conventional chemotherapies such as bortezomib. Moreover, the safety profile of the masitinib plus (bortezomib/dexamethasone) combination appears to be superior to those combinations of masitinib plus (dexamethasone/bortezomib/thalidomide), or masitinib plus (dexamethasone/bortezomib/adriamycin). Significantly, the combination of masitinib plus (bortezomib/dexamethasone) was well tolerated at the standard dose of bortezomib in this indication. It was also discovered that superior efficacy was achieved for VD level 2 (four times bortezomib and six times dexamethasone per cycles) than for VD level 1 (two times bortezomib and four times dexamethasone per cycles) with similar tolerable toxicity profiles. Comparison of data from patients in second-line treatment (first relapse) and patients beyond second-line treatment (beyond first relapse), suggest that masitinib in combination with other anti-myeloma agents is preferentially administered to early stage or first relapsed multiple myeloma patients.

### OVERALL CONCLUSIONS

These two clinical studies and preclinical data provide evidence that a tyrosine kinase inhibitor or a mast cell inhibitor, especially masitinib or a pharmaceutically acceptable salt thereof, is a viable therapeutic strategy for multiple myeloma, especially for the treatment of refractory or first relapsed multiple myeloma, and in particular patients with t(4;14) multiple myeloma, with both positive and negative FGFR3 expression mutation status. Masitinib demonstrated significant activity against FGFR3 negative multiple myeloma as well as for FGFR3 positive multiple myeloma, indicating that the FGFR3 inhibition is not a dominant mechanism of action behind the observed therapeutic effect of masitinib. Other multiple myeloma associated kinases targeted by masitinib (for example, c-Kit, Lyn, and PDGFR) are inhibited at submicromolar concentrations. Similarly, masitinib's potentiating effect on dexamethasone was achievable at lower concentrations than that required to inhibit FGFR3. These concentrations are reachable in vivo with a masitinib dose of 6 mg/kg/day. Therefore, unexpectedly, the starting dose at which masitinib can provide a therapeutic benefit can be lowered to 6.0 mg/kg/day ±1.5 mg/kg/day. This will reduce the risk of toxicity at the start of treatment while still delivering a clinically relevant dose. Moreover, this realization that masitinib's mechanism of action in multiple myeloma is strongly connected to its inhibition of kinases other than FGFR3, and most likely with its potent action on mast cell proliferation, survival and activation, leads to the conclusion that a tyrosine kinase inhibitor or a mast cell inhibitor, especially masitinib, optionally administered in combination with at least one other anti-myeloma agent, can also provide therapeutic benefit in early stage treatment, such as a first-line adjuvant therapy or part of a maintenance therapy regimen.

1. Use of a tyrosine kinase inhibitor or a mast cell inhibitor for the preparation of a medicament for the treatment of multiple myeloma in human patients, wherein said tyrosine kinase inhibitor or mast cell inhibitor is administered in association with an additional care in multiple myeloma.

2. The use according to claim 1 wherein said tyrosine kinase inhibitor or a mast cell inhibitor is masitinib or a pharmaceutically acceptable salt thereof.

3. The use according to claim 1 or 2, wherein said treatment of multiple myeloma is treatment of refractory or first relapsed multiple myeloma.

4. The use according to any one of the preceding claims, wherein said human patients are patients with t(4;14) multiple myeloma.

5. The use according to any one of the preceding claims, wherein, said additional care is selected from the group consisting of autologous stem-cell transplantation, targeted therapies and anti-myeloma agents.

6. The use according to any one of the preceding claims, wherein said anti-myeloma agents are selected from the group consisting of alkylating agents, corticosteroids, or immunomodulatory agents.

7. The use according to any one of the preceding claims, wherein, said anti-myeloma agents are selected from the group consisting of bortezomib, lenalidomide, and dexamethasone.

8. The use according to any one of the preceding claims, wherein said patients are those afflicted by t(4;14) multiple myeloma with expression of FGFR3.

9. The use according to claim 1, wherein said patients are those afflicted by t(4;14)-negative multiple myeloma.

10. The use according to any one of the preceding claims, wherein said patients are those afflicted by multiple myeloma with expression of c-Kit.

11. The use according to claim 10, wherein said patients are those afflicted by multiple myeloma with expression of c-Kit GNNK-negative form.

12. The use according to any one of the preceding claims, wherein said tyrosine kinase inhibitor or a mast cell inhibitor is administered for the treatment of relapsing multiple myeloma, as defined by the International uniform response criteria for multiple myeloma (International Myeloma Working Group criteria), in patients who received one previous therapy.

13. The use according to any one of the claims 1 to 11, wherein said tyrosine kinase inhibitor or a mast cell inhibitor is administered for the treatment of refractory multiple myeloma.

14. The use according to claim 13 wherein said tyrosine kinase inhibitor or a mast cell inhibitor is administered for the treatment of refractory multiple myeloma in patients resistant to bortezomib, lenalidomide, and/or dexamethasone.

15. The use according to any one of the preceding claims, wherein said tyrosine kinase inhibitor or a mast cell inhibitor is masitinib mesilate.

16. The use according to any one of the preceding claims, wherein said tyrosine kinase inhibitor or a mast cell inhibitor is a dual c-Kit/FGFR3 inhibitor.

17. The use according to any one of the preceding claims, wherein said tyrosine kinase inhibitor or a mast cell inhibitor is an inhibitor of c-Kit, PDGFR, Lyn and Fyn kinase activity.

18. The use according to any one of the preceding claims, wherein said tyrosine kinase inhibitor or a mast cell inhibitor is masitinib or a pharmaceutically acceptable salt thereof, which is administered at a starting daily dose of 3.0 to 9.0 mg/kg/day.

19. The use according to any one of the preceding claims, wherein said patients are patients with refractory or first relapsed multiple myeloma and wherein said tyrosine kinase inhibitor or a mast cell inhibitor is masitinib or a pharmaceutically acceptable salt thereof, which is administered at a starting daily dose of 6.0 mg/kg/day ±1.5 mg/kg/day.

20. The use according to any one of the preceding claims, wherein said tyrosine kinase inhibitor or a mast cell inhibitor is masitinib or a pharmaceutically acceptable salt thereof, which is dose escalated by increments of 1.5 mg/kg/day to reach a maximum of 12.0 mg/kg/day.

21. The use according to any one of the preceding claims, wherein said tyrosine kinase inhibitor or mast cell inhibitor is administered orally.

22. The use according to any one of the preceding claims, wherein said tyrosine kinase inhibitor or mast cell inhibitor is administered twice a day.

23. The use according to any one of the preceding claims comprising a long-term administration of an effective amount of said tyrosine kinase inhibitor or mast cell inhibitor, over more than 3 months.

24. The use according to claim 23, wherein said long-term administration is over more than 12 months.

25. The use according to any one of the preceding claims, wherein the said pharmaceutical composition comprises a dose of at least 50 mg and less than 150 mg of said tyrosine kinase inhibitor or mast cell inhibitor.

26. The use according to any one of claims 1 to 24, wherein the said pharmaceutical composition comprises a dose of at least 150 mg and less than 400 mg of said tyrosine kinase inhibitor or mast cell inhibitor.

27. The use according to any one of the preceding claims wherein the tyrosine kinase inhibitor or a mast cell inhibitor is masitinib or a pharmaceutically acceptable salt thereof, and said patients are patients with t(4;14)-positive multiple myeloma.

28. The use according to any one of the preceding claims wherein the tyrosine kinase inhibitor or a mast cell inhibitor is masitinib or a pharmaceutically acceptable salt thereof and said patients are patients with t(4;14)-negative multiple myeloma.

29. The use according to any one of the preceding claims wherein the tyrosine kinase inhibitor or a mast cell inhibitor is masitinib or a pharmaceutically acceptable salt thereof and said patients are patients with c-Kit expression.

30. The use according to any one of the preceding claims wherein said medicament is an adjuvant or maintenance therapy and wherein said treatment of multiple myeloma is prevention of relapse following treatment-induced remission or post-autologous stem-cell transplantation.

31. The use according to claim 30, wherein said additional care is at least one other anti-myeloma agent.

32. The use according to any one of the preceding claims wherein said treatment is first-line treatment of multiple myeloma in combination with at least one other anti-myeloma agent.

33. The use according to any one of the preceding claims wherein said treatment is second-line treatment of multiple myeloma in combination with at least one other anti-myeloma agent.

34. The use according to claim 32 or 33, wherein said at least one other anti-myeloma agent is selected from the group consisting of targeted therapy agent, alkylating agent, corticosteroid, or immunomodulatory agent.

35. The use according to claim 34, wherein said at least one other anti-myeloma agent is selected from the group consisting of bortezomib, lenalidomide, and dexamethasone.

36. The use according to any one of the preceding claims wherein said treatment is treatment of refractory multiple myeloma, and wherein said tyrosine kinase inhibitor or a mast cell inhibitor is in combination with at least one other anti-myeloma agent.

37. The use according to claim 36, wherein said anti-myeloma agent is selected from the group consisting of targeted therapy agent, alkylating agent, corticosteroid, and immunomodulatory agent.

38. The use according to any one of the preceding claims wherein said additional care is at least one agent selected from the group consisting of bortezomib, dexamethasone, thalidomide, lenalidomide, doxorubicin, vincristine, melphalan, cyclophosphamide, pomalidomide, carfilzomib, elotuzumab, vorinostat, and panabinostat.

39. The use according to any one of the preceding claims wherein said additional care is at least one anti-myeloma agent and wherein said tyrosine kinase inhibitor or mast cell inhibitor and at least one anti-myeloma agent are administered separately, simultaneously or sequentially in time.
